(19) United States
(12) Patent Application Publication
(10) Pub. No.: US 2012/0064516 A1
(43) Pub. Date: Mar. 15, 2012

(54) NUCLEIC ACID AMPLIFICTION REACTION APPARATUS, SUBSTRATE FOR NUCLEIC ACID AMPLIFICATION REACTION APPARATUS, AND NUCLEIC ACID AMPLIFICATION REACTION METHOD

(75) Inventor: Junji Kajihara, Tokyo (JP)

(73) Assignee: SONY CORPORATION, Tokyo (JP)

(21) Appl. No.: 13/227,661

(22) Filed: Sep. 8, 2011

(30) Foreign Application Priority Data
Sep. 15, 2010 (JP) 2010-206752

(51) Int. Cl. C12M 1/40; C12Q 1/68

(52) U.S. Cl. 435/6.1; 435/289.1

(57) ABSTRACT

A nucleic acid amplification reaction apparatus includes: a reaction region formed as a nucleic acid amplification reaction site in the shape of a tapered well of a decreasing horizontal cross sectional area along a light axis direction in which a substance that precipitates in the course of a nucleic acid amplification reaction settles; temperature controller that heats the reaction region; irradiator that shines light on the reaction region; and detector that detects the quantity of the light scattered out of the reaction region by the precipitated substance.

### CROSS REFERENCES TO RELATED APPLICATIONS

The present application claims priority to Japanese Priority Patent Application JP 2010-206752 filed in the Japan Patent Office on Sep. 15, 2010, the entire content of which is hereby incorporated by reference.

### BACKGROUND

The present application relates to nucleic acid amplification reaction apparatuses, substrates for nucleic acid amplification reaction apparatuses, and nucleic acid amplification reaction methods. Specifically, the present application concerns a nucleic acid amplification reaction apparatus that includes reaction regions of a tapered well shape formed as the reaction sites of a nucleic acid amplification reaction.

Gene amplification by methods such as PCR (Polymerase Chain Reaction) and LAMP (Loop-Mediated Isothermal Amplification) represents the standard technique for the quantitative analysis of trace amounts of nucleic acid. The technique has been used for gene expression analyses, and for testing of hereditary diseases, cancers, and microbial and viral infections. The technique is also used for gene analyses such as SNP analysis.

Various methods are known as the common methods of detecting nucleic acid amplification products, including fluorescence detection methods that use fluorescent substances such as in the intercalator method and the fluorescence-labeled probe method, and a turbidity detection method that uses a turbidity substance formed as water insoluble or poorly soluble salts from metal ions such as magnesium ions that bind to the pyrophosphoric acid formed as a by-product in the process of amplification, as described in JP-A-2008-237207, WO/2001/83817, and Japanese Patent No. 413347.

Nucleic acid amplification reaction apparatuses that use nucleic acid amplification product detection methods such as above for gene expression analyses, infection testing, and gene analyses such as SNP analysis are widely available in the market.

### SUMMARY

These nucleic acid amplification reaction apparatuses typically use a columnar well plate, and further improvements in detection sensitivity are needed.

Accordingly, there is a need for a nucleic acid amplification reaction apparatus, a substrate for nucleic acid amplification reaction apparatuses, and a nucleic acid amplification reaction method with which improved detection sensitivity can be obtained.

An embodiment is directed to a nucleic acid amplification reaction apparatus that includes: a reaction region formed as a nucleic acid amplification reaction site in the shape of a tapered well of a decreasing horizontal cross sectional area along a light axis direction in which a substance that precipitates in the course of a nucleic acid amplification reaction settles; temperature controller that heats the reaction region; irradiator that shines light on the reaction region; and detector that detects the quantity of the light scattered out of the reaction region by the precipitated substance.

It is preferable that the reaction region have a slanted inner surface subjected to a smoothing treatment.

Another embodiment is directed to a microchip for nucleic acid amplification reaction that includes a reaction region formed as a nucleic acid amplification reaction site, wherein the reaction region is a tapered well of a decreasing cross sectional area along a light axis direction in which a substance that precipitates in the course of a nucleic acid amplification reaction settles.

Still another embodiment is directed to a nucleic acid amplification reaction method that includes shining light on a reaction region provided as a nucleic acid amplification reaction site, and detecting the quantity of the light scattered by a substance that precipitates in the course of an amplification reaction, the reaction region being a tapered well of a decreasing cross sectional area along a light axis direction in which the substance that precipitates in the course of the nucleic acid amplification reaction settles.

Improved detection sensitivity can be obtained with the nucleic acid amplification reaction apparatus, the substrate, and the nucleic acid amplification reaction method provided by the embodiments.

Additional features and advantages are described herein, and will be apparent from the following Detailed Description and the figures.

### BRIEF DESCRIPTION OF THE FIGURES

FIG. 1 is a schematic diagram illustrating a nucleic acid amplification reaction apparatus according to an embodiment.

FIGS. 2A and 2B are vertical sectional views of a reaction region of a microchip for nucleic acid amplification reaction according to an embodiment, taken along the light axis direction.

### DETAILED DESCRIPTION

Embodiments of the present application will be described below in detail with reference to the drawings.

1. Nucleic acid amplification reaction apparatus

(1) Reaction regions

(a) Substrate (microchip for nucleic acid amplification reaction)

(b) Nucleic acid amplification reaction

(c) Nucleic acid amplification (product) detection method

(2) Temperature controller

(3) Irradiator

(4) Detector

2. Operation of nucleic acid amplification reaction apparatus (1) Variations

(a) Operation of RT-LAMP apparatus

(b) Operation of RT-PCR apparatus

1. Nucleic Acid Amplification Reaction Apparatus

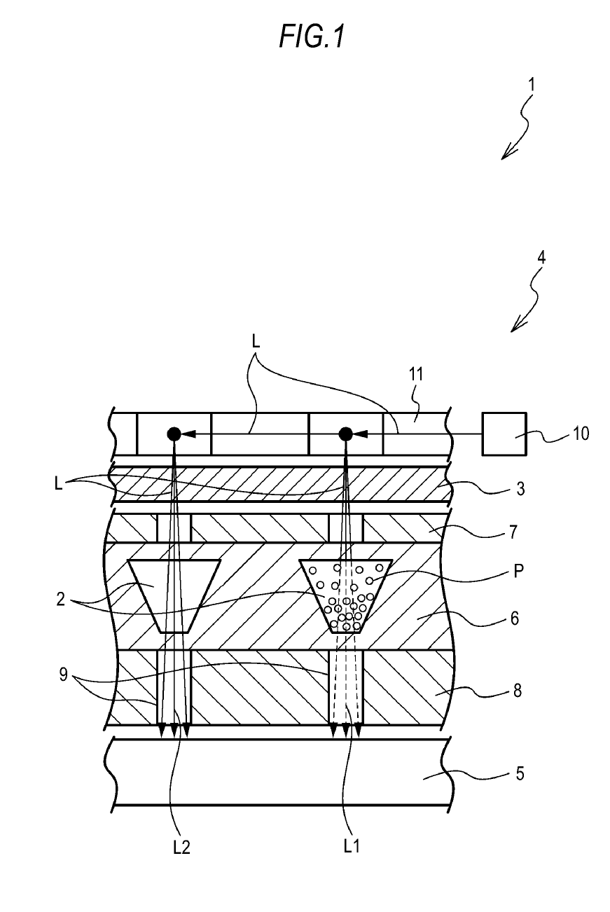

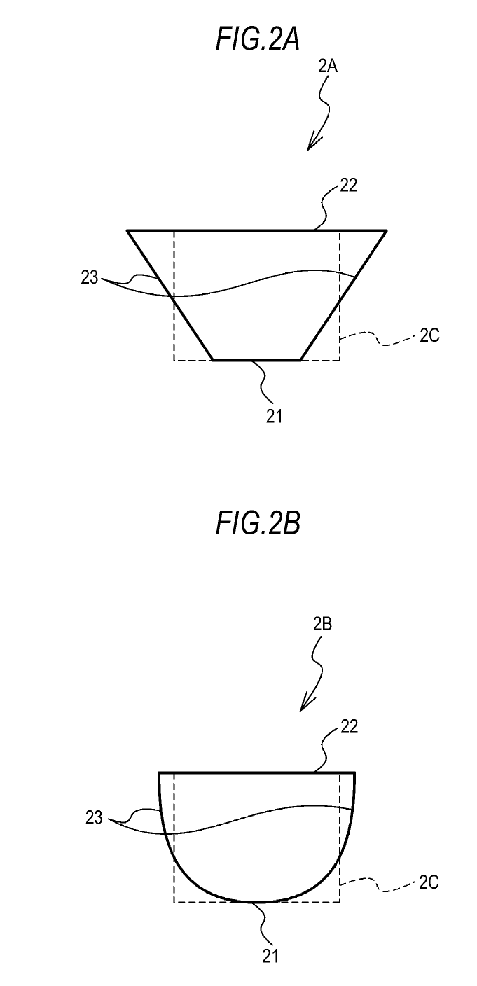

FIG. 1 is a schematic diagram of a nucleic acid amplification reaction apparatus according to an embodiment. FIGS. 2A and 2B are vertical sectional views of a reaction region of a microchip for nucleic acid amplification reaction according to an embodiment, taken along the light axis direction.

Note that the configuration and other details of the apparatus illustrated in the figures are simplified for ease of explanation.

A nucleic acid amplification reaction apparatus 1 according to the embodiment is operable to control a nucleic acid amplification reaction for the amplification and quantification of nucleic acid using reaction regions 2, a temperature controller 3, an irradiator 4, and a detector 5.

In the nucleic acid amplification reaction apparatus 1 of the embodiment, the temperature controller 3 and the reaction regions 2 (detachably provided; substrate 6) are disposed between the irradiator 4 and the detector 5.

Further, pin holes 7, a filter (not illustrated), and a condensing lens (not illustrated) also may be appropriately provided between the reaction regions 2 and the irradiator 4 for adjustments of, for example, light quantity and light component. Further, a substrate support 8, a filter (not illustrated), and a condensing lens (not illustrated) may be appropriately provided between the reaction regions 2 and the detector 5 for supporting the reaction regions 2 and for adjustments of, for example, light quantity and light component.

The nucleic acid amplification reaction apparatus 1 according to the embodiment also includes a control unit (not illustrated) that controls various operations concerning the apparatus of the embodiment (including, for example, light control, temperature control, nucleic acid amplification reaction, detection control, calculations of detected light quantity, and monitoring).

The configuration of each element is described below in detail.

(1) Reaction Regions

The reaction regions 2 are areas provided as the reaction sites of a nucleic acid amplification reaction, and are formed into a tapered well shape.

The tapered wells are shaped so that the horizontal cross sectional area of the reaction regions becomes smaller along the light axis direction in which the substance (precipitate) P that precipitates in the course of a nucleic acid amplification reaction settles.

In a common well, for example, a columnar well, the light traveling distance in the reaction region, specifically the well length must be extended along the light axis direction to improve detection sensitivity or detection accuracy in the low scattering state at early stages of reaction. However, extending the well increases the distance from the heat source that controls the temperature of the nucleic acid amplification reaction. This creates large temperature differences inside the well, and tends to lower detection stability by lowering the efficiency of nucleic acid amplification reaction or by causing nonuniform reactions.

However, with the reaction regions formed into a tapered well shape as in the embodiment, the substance that precipitates and settles at the reaction sites is more concentrated toward the bottom surface of the wells, and the extent of scattering increases. Specifically, the tapered well shape enables controlling the scattering cross sectional area of the light passage region, and thereby locally increases the agglomeration degree of the precipitate in the horizontal cross sectional area of the reaction regions. In this way, even trace amounts of nucleic acid in the reaction regions can be detected without increasing the light traveling distance. Further, detection sensitivity at early stages of reaction (low scattering) improves, and monitoring becomes desirable.

In this manner, the tapered well shape of the reaction regions desirably improves the detection sensitivity and detection accuracy of the turbidity detection through the nucleic acid amplification reaction. Further, the efficiency of turbidity detection can be improved with a microchip for nucleic acid amplification reaction provided with the tapered well according to the embodiment. Further, because the performance and reliability of the apparatus can be ensured without increasing the scattering distance, the overall size, particularly the thickness, of the nucleic acid amplification reaction apparatus can be reduced with ease.

FIGS. 2A and 2B are examples of the cross sectional shape of one of the reaction regions 2 taken along the vertical flat plane through the center of a bottom surface 21 of the reaction region 2. The vertical sectional shape of a columnar well 2C is indicated by broken lines.

The reaction region 2 has a top surface 22 and the bottom surface 21, which are preferably flat surfaces that allow for passage of light in the same light axis direction. Preferably, these two surfaces are a pair of parallel opposed surfaces.

The reaction region 2 with the bottom surface 21 and the top surface 22 also has a slanted surface or surfaces 23 that help the precipitate P to settle. Preferably, the slanted surfaces 23 of the reaction region 2 are shaped in such a manner that the width of the reaction region 2 becomes smaller toward the bottom surface 21 in the vertical section taken along the light axis direction. Examples of such shapes include a truncated pyramid shape 2A and a concave paraboloidal shape 2B.

The inner surfaces (slanted surfaces 23) of the reaction region 2 may form, for example, a frustum such as a circular truncated cone and a polygonal frustum, or a paraboloid of revolution about the light axis. Of these, the circular truncated cone is preferred for ease of formation. Detection sensitivity and detection accuracy can be improved by controlling the scattering cross sectional area of the light passage region formed by the tapered slanted surfaces 23, even when the light passage region has the same volume and the same light traveling distance as the columnar well 2C. Specifically, the area of the bottom surface 21 of the reaction region 2 is preferably 1/2 to 1/5, more preferably 1/3 to 1/4 of the area of the top surface 22. With the area of the bottom surface 21 set within this area ratio, a slope can be formed that helps the precipitate P to settle without being adsorbed on the slanted surfaces 23. Because the agglomeration degree of the precipitate can efficiently increase locally on the bottom surface side, the detection sensitivity and detection accuracy of the turbidity detection through the nucleic acid amplification reaction become desirable.

Preferably, the inner surfaces of the slanted surfaces 23 of the reaction region 2 are subjected to a smoothing treatment. The smoothing treatment further helps prevent the precipitate (particles) P from adhering to the slanted surfaces, and makes the slope more effective at producing a higher precipitate P concentration toward the bottom surface side of the well. This makes the detection sensitivity and detection accuracy of the turbidity detection even more desirable.

The smoothing treatment may be, for example, polishing or coating. Polishing may be performed by, for example, chemical mechanical polishing using a polisher. Examples of polisher include inorganic filler (particles)-containing slurry polishers used to polish plastic substrates and glass substrates. The inorganic filler may be one or more selected from, for example, calcium carbonate, aluminum hydroxide, calcium hydroxide, magnesium hydroxide, titanium oxide, silicic anhydride, and silica stone.

The coating agent used for coating may be, for example, silicon, and silicons with high transmissivity can preferably used advantageously. Silicons with high transmissivity can be applied to the whole inner surfaces of the reaction regions 2, and thus desirably improves the production efficiency of the microchip for nucleic acid amplification reaction, specifically the substrate that includes the reaction regions 2.

(a) Substrate (Microchip for Nucleic Acid Amplification Reaction)

A reaction chamber (for example, a substrate), for example, such as a microchip for nucleic acid amplification reaction preferably includes one or more reaction regions 2. The tapered well shape can easily be formed in such a reaction chamber.

The microchip for nucleic acid amplification reaction (substrate 6) provided with the reaction regions 2 can be formed from a single or a plurality of substrates.

One of the end surfaces of the substrate 6 is the surface irradiated with light (top surface 22 side), and the other end surface is the surface through which the light passing through the reaction regions (optical path in the reaction sites) emerges (bottom surface 21 side). Preferably, these surfaces represent a pair of parallel opposed surfaces.

The top surface 22 of the reaction regions 2 is irradiated with light, which then passes through the reaction regions 2 and emerges through the bottom surface 21. The detector 5 then detects the quantities of scattered light and transmitted light. Note that the quantities of scattered light and transmitted light also may be measured by shining light from the side on the bottom surface side of the reaction regions 2 of the microchip for nucleic acid amplification reaction.

The method used to form the reaction regions 2 in the substrate 6 is not particularly limited. Preferably, the reaction regions 2 are formed, for example, by the wet etching or dry etching of a glass substrate layer, or by the nanoimprinting, injection molding, or cutting of a plastic substrate layer.

For example, one or more reaction regions 2 of, for example, the truncated pyramid shape 2A or the concave paraboloidal shape 2B may be formed in a single substrate by abrasive cutting or molding, and another substrate may be disposed on the top surface of this substrate.

The material of the substrate 6 is not particularly limited, and is appropriately selected by taking into consideration such factors as the detection method, ease of processing, and durability. The material is appropriately selected from light transmissive materials according to the desired method of detection, for example, from glass materials and various plastics (such as polypropylene, polycarbonate, cycloolefin polymer, and polydimethylsiloxane).

Reagents necessary for the nucleic acid amplification reaction may be stored beforehand in the reaction regions 2 formed as above.

(b) Nucleic Acid Amplification Reaction

As used herein, “nucleic acid amplification reaction” encompasses both PCR (polymerase chain reaction) reactions that involve temperature cycles, and various isothermal amplification reactions that do not involve temperature cycles. Examples of isothermal amplification reactions include LAMP (Loop-Mediated Isothermal Amplification), SMAP (SMartAmplification Process), NASBA (Nucleic Acid Sequence-Based Amplification), ICAN® (Isothermal and Chimeric primer-initiated Amplification of Nucleic acids), a TRC (transcription-reverse transcription concerted) method, SDA (strand displacement amplification), TMA (transcription-mediated amplification), and RCA (rolling circle amplification).

The “nucleic acid amplification reaction” also includes a wide range of nucleic acid amplification reactions involving or not involving temperature changes and intended for nucleic acid amplification. The “nucleic acid amplification reaction” also includes reactions that involve quantification of the amplified nucleic acid chain, such as real-time PCR (RT-PCR) and RT-LAMP.

The term “reagent” is that required for obtaining amplified nucleic acid chains in the nucleic acid amplification reaction. Specific examples include oligonucleotide primers having the base sequences complementary to the target nucleic acid chains, nucleic acid monomer (dNTP), enzymes, and reaction buffer (buffer) solutes.

The PCR method involves the continuous amplification cycles of heat denaturation (about 95° C.) primer annealing (about 55 to 60° C.) extension reaction (about 72° C.).

The LAMP method is a technique that takes advantage of DNA loop formation to obtain the amplification product dsDNA from DNA or RNA at constant temperature. As an example, the reaction proceeds with addition of components (i), (ii), and (iii), upon which the inner primer forms stable base pairs with the complementary sequence on the template nucleic acids, and the mixture is incubated at a temperature that can maintain the enzyme activity of the strand-displacing polymerase. The incubation temperature is preferably 50 to 70° C., and the incubation time is preferably about 1 min to about 10 hours.

Component (i): Two inner primers, additionally with two outer primers or two loop primers

Component (ii): Strand-displacing polymerase

Component (iii): Substrate nucleotides

(c) Nucleic Acid Amplification (Product) Detection Method

The nucleic acid amplification detection method may be performed by methods using, for example, a turbidity substance, a fluorescent substance, or a chemiluminescent substance.

The method using a turbidity substance may be a method that uses, for example, the precipitates formed by the pyrophosphoric acid resulting from the nucleic acid amplification reaction, and metal ions that can bind to the pyrophosphoric acid. The metal ions are monovalent or divalent metal ions, and become a turbidity substance in the form of water insoluble or poorly soluble salts formed upon the binding of the metal ions to the pyrophosphoric acid.

Specific examples of such metal ions include alkali metal ions, alkali earth metal ions, and divalent transition metal ions. Preferably, the metal ions are one or more selected from, for example, alkali earth metal ions (such as magnesium(II), calcium(II), and barium(II)), and divalent transition metal ions (such as zinc(II), lead(II), manganese(II), nickel(II), and iron(II)). Magnesium(II), manganese(II), nickel(II), and iron(II) are more preferred.

The preferred concentration before the addition of the metal ions ranges form 0.01 to 100 mM. The detection wavelength is preferably from 300 nm to 800 nm.

The method using a fluorescent substance or a chemiluminescent substance may be, for example, an intercalation method that uses a fluorescent dye (derivative) that fluoresces by being specifically inserted to double-stranded nucleic acids, or a labeled probe method that uses a probe formed by bonding a fluorescent dye to an oligonucleotide specific to the amplified nucleic acid sequence.

Examples of the labeled probe method include a hybridization (Hyb) probe method, and a hydrolysis (TaqMan) probe method.

The Hyb probe method is a method that uses two probes, a donor dye-labeled probe and an acceptor dye-labeled probe, designed to come close to each other. The donor dye excites the acceptor dye, and the acceptor dye fluoresces upon the two probes hybridizing with the target nucleic acid.

The TaqMan probe method is a method that uses a probe labeled with a reporter dye and a quencher dye in proximity to each other. The quencher dye and the reporter dye separate from each other as the probe is hydrolyzed during the nucleic acid extension, and the reporter dye fluoresces upon being excited.

Examples of the fluorescent dye (derivative) used in the methods that use fluorescent substances include SYBR® Green I, SYBR® Green II, SYBR® Gold, YO (Oxazole Yellow), TO (Thiazole Orange), PG (Pico® Green), and ethidium bromide.

Examples of the organic compound used in the methods that use chemiluminescent substances include luminol, lophine, lucigenin, and oxalate.

(2) Temperature Controller

The temperature controller 3 is provided to heat the reaction regions 2. The temperature controller 3 is not particularly limited, and, for example, a Peltier heater and a light-transmissive ITO heater can be used.

The temperature controller 3 may have a shape of, for example, a thin film or a flat plate.

Preferably, the temperature controller 3 is disposed at a position that allows the heat to readily transfer to the reaction regions 2. For example, the temperature controller 3 is preferably disposed close to the reaction regions 2. Specifically, the temperature controller 3 may be disposed anywhere near the reaction regions 2, including above, beneath, and sides of the reaction regions 2, and around the reaction regions 2. Other members, for example, such as the pin holes 7 may be interposed in between.

Preferably, the temperature controller 3 has a shape of a thin film or a flat plate, and is disposed above and/or beneath the reaction regions 2. Here, the temperature controller 3 may be disposed as the substrate support 8, and holes 9 may be provided on the light axis to transmit light. Because there is no need to increase the light traveling distance in the reaction regions 2, the distance from the heat source does not become long, and the temperature inside the reaction regions 2 can be easily controlled. As a result, the detection sensitivity and detection accuracy of the turbidity detection improve.

(3) Irradiator

The irradiator 4 is configured to include one or more light sources 10, and to irradiate the reaction regions 2 with light L emitted by the light source 10. Specifically, the irradiator 4 is configured to irradiate the top surface 22 of the reaction regions 2 with the light L emitted by the light source 10, so as to detect the quantity of the light scattered by the precipitate P formed in the course of the nucleic acid amplification reaction. For example, the light source 10 may be disposed above the top surface 22 of the reaction regions 2, or a light guide 11 that guides the emitted light L from the light source 10 to the reaction regions 2 may be disposed.

Preferably, the irradiator 4 includes the light guide 11 that allows the emitted light from the light source 10 to be shone on the reaction regions 2. The light guide 11 has a light incident end on which the light emitted by one or more light sources 10 is incident. Members (for example, a prism, a reflector, and irregularities) that guide the incident light L to each reaction region are provided inside the light guide 11.

By the provision of the light guide 11, the number of light sources can be reduced, and the light can be uniformly shone onto one or more of the reaction regions 2 over the substrate 6. Further, the detection sensitivity and detection accuracy of the turbidity detection can be desirably improved. The fewer light sources enable the overall size, particularly the thickness of the apparatus to be reduced, and can also reduce power consumption.

The light source 10 is not particularly limited, and is preferably one that desirably emits light that can be used to desirably detect the target nucleic acid amplification product. Examples of the light source 10 include laser light sources, white or monochromatic light-emitting diodes (LEDs), a mercury lamp, and a tungsten lamp. Of these, LEDs are preferred in terms of power consumption and cost. LEDs are also advantageous for their ability to produce a desired light component with the use of various filters.

The type of laser used for the laser light source is not particularly limited, provided that the light source emits, for example, an argon ion (Ar) laser, a helium-neon (He-Ne) laser, a dye laser, and a krypton (Cr) laser. One or more laser light sources may be freely combined and used.

As illustrated in FIG. 1, the light L from the irradiator 4 reaches the reaction regions 2, and is reflected or absorbed by the precipitate (generated turbidity substance) P formed in the course of the amplification reaction in the reaction regions. The quantity of the light scattered by the turbidity substance P, or the quantity of transmitted light (light L1, L2) is then appropriately detected by the detector 5 (optical detector) through elements such as an aperture (holes 9), a condensing lens, and a fluorescence filter.

The scattered light may be, for example, forward-scattered light, backscattered light, or side scattered light. In the present apparatus, forward-scattered light is advantageous, because it can be easily detected with good detection sensitivity.

(4) Detector

The detector 5 is a mechanism capable of detecting the quantity of the light L1 and L2 emitted from the end of the reaction regions 2 (specifically, from the bottom surface 21). The detector 5 includes at least an optical detector.

The optical detector is not particularly limited, and may be, for example, a photodiode (PD) array, an area imaging device (such as a CCD image sensor and a CMOS image sensor), a small light sensor, a line sensor scanner, or a PMT (photomultiplier tube), which may be used in appropriate combinations. The optical detector detects material such as the turbidity substance P produced by the nucleic acid amplification reaction.

An excitation filter or a fluorescence filter may be appropriately disposed inside the nucleic acid amplification reaction apparatus 1 of the embodiment. For example, an excitation filter may be disposed between the irradiator 4 and the reaction regions 2, or a fluorescence filter may be disposed between the reaction regions 2 and the detector 5.

With the excitation filter (not illustrated), a light component of a desired specific wavelength can be obtained, or unnecessary light components can be removed, according to the detection method used for the nucleic acid amplification reaction. With the fluorescence filter (not illustrated), light components necessary for the detection (scattered light, transmitted light, fluorescence) can be produced. In this way, detection sensitivity and detection accuracy can be improved.

2. Operation of Nucleic Acid Amplification Reaction Apparatus 1

The following describes the operation of the nucleic acid amplification reaction apparatus 1, and the nucleic acid amplification reaction method that detects the quantity of the light scattered by the turbidity substance P.

The light source 10 emits light L. The light L is incident on the light guide 11 through the light incident end. The incident light L is then guided onto the incident end (top surface 22) of the reaction regions 2 by the prism and other members provided inside the light guide 11.

The light L falls on one end of the reaction regions 2 (top surface 22) of a tapered well shape formed as the reaction sites of the nucleic acid amplification reaction, and enters the tapered wells. Here, the precipitate P formed by the nucleic acid amplification reaction is concentrated by the slanted surfaces 23 toward the bottom surface side of the wells, and the extent of light scattering increases. The light L shines the precipitate P produced in the course of the nucleic acid amplification reaction in the reaction regions 2. The light L that falls on the precipitate P is reflected or absorbed at the surface of the precipitate P inside the reaction regions 2, and turns into light L1 (scattered light and transmitted light). Light L2 occurs when the amount of precipitate P is small. The light L1 and L2 emerge from the other end of the reaction regions 2 (bottom surface 21). The emitted light L1 and L2 may be appropriately passed through a fluorescence filter to produce a desired light component (for example, a scattered light component or a transmitted light component). The quantities of the emitted light L 1 and L2 are then detected by the detector 5 (optical detector). Specifically, the quantity of the light scattered by the precipitate P produced in the course of the amplification reaction is detected.

It is thus preferable that the turbidity detection through nucleic acid amplification reaction proceed by irradiation of the reaction regions formed into a tapered well shape, and by detection of the quantity of the light scattered by the substance precipitated in the course of the reaction.

Further, as described above, the reaction regions are preferably of a tapered well shape with a decreasing cross sectional area along the light axis direction in which the precipitate formed in the course of the nucleic acid amplification reaction settles.

In this way, the agglomeration degree of the precipitate formed by the binding of metal ions to the pyrophosphoric acid produced by the nucleic acid amplification increases more toward the bottom surface side of the wells, without having the need to increase the light traveling distance. Because the quantity of the light scattered by the precipitate (or the quantity of transmitted light) more easily decreases, detection sensitivity and detection accuracy can improve.

Preferably, the slanted inner surfaces of the reaction regions are subjected to a smoothing treatment. It is also preferable that the inner surfaces of the reaction regions form a circular or polygonal frustum, or a concave paraboloid of revolution. Further, it is preferable that the bottom surface area of the reaction regions is 1/2 to 1/5 of the top surface area. It is also preferable that the detection through the nucleic acid amplification reaction be performed by turbidity detection using a LAMP or a PCR method. In this way, the precipitate is concentrated more toward the bottom surface side of the wells, making it possible to desirably improve detection sensitivity and detection accuracy.

(1) Variations

The nucleic acid amplification reaction apparatus of the embodiment also may be used as a nucleic acid amplification detection apparatus by installing the reaction regions 2 in, for example, the temperature controller 3 after the reaction.

The nucleic acid amplification reaction apparatus also may be used as a LAMP apparatus or a PCR apparatus to quantify nucleic acid by turbidity substance detection.

(a) Operation of RT-LAMP Apparatus

A nucleic acid detection method using a RT-LAMP apparatus is described below based on step S11.

In the temperature control step (step S11), the nucleic acid in each reaction region 2 is amplified under the constant temperature (60 to 65° C.) set for the reaction regions 2. It should be noted that the LAMP method does not require the heat denaturation from double strands to single strands, and the primer annealing and nucleic acid extension are repeated under the isothermal conditions.

The nucleic acid amplification reaction produces pyrophosphoric acid, which binds to metal ions and forms insoluble or poorly soluble salts as the turbidity substance (measurement wavelength, 300 nm to 800 nm). Incident light (light L) falls on the turbidity substance and is scattered (light L1, L2). The detector 5 then measures the quantity of the scattered light in real time for quantification. Quantification also may be made from the quantity of transmitted light.

(b) Operation of RT-PCR Apparatus

A nucleic acid detection method using a RT-PCR apparatus is described below based on step Sp1 (heat denaturation), step Sp2 (primer annealing), and step Sp3 (DNA extension).

In the heat denaturation step (step Sp1), double-stranded DNA is denatured into single-stranded DNA in the reaction regions 2 set to 95° C. under the control of the temperature controller.

In the next annealing step (step Sp2), the primers bind to the complementary base sequences of the single-stranded DNA in the reaction regions 2 set to 55° C.

In the DNA extension step (step Sp3), the reaction regions 2 are controlled at 72° C. to extend cDNA by the polymerase reaction that proceeds from the primers as the origin of DNA synthesis.

The DNA in each reaction region 2 is amplified by the repeated temperature cycles of steps Sp1 to Sp3. The nucleic acid amplification reaction produces pyrophosphoric acid, and the turbidity substance is detected and the nucleic acid amount is quantified as above.

The nucleic acid amplification reaction apparatus according to the embodiment enables measurements with high detection sensitivity. This is made possible by the tapered well shape of the reaction regions of a decreasing horizontal cross sectional area, which locally increases the agglomeration degree of the precipitate on the bottom surface side of the wells. Further, because there is no need to increase the light traveling distance, the overall size of the apparatus can be reduced, and, particularly, the thickness can be reduced to make the apparatus handy. Fluorescence detection is also possible as may be required.

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

1. A nucleic acid amplification reaction apparatus, comprising:
a reaction region formed as a nucleic acid amplification reaction site in the shape of a tapered well of a decreasing horizontal cross sectional area along a light axis direction in which a substance that precipitates in the course of a nucleic acid amplification reaction settles;
temperature control means that heats the reaction region;
irradiating means that shines light on the reaction region; and
detecting means that detects the quantity of the light scattered out of the reaction region by the precipitated substance.

2. The apparatus according to claim 1, wherein the reaction region has a slanted inner surface subjected to a smoothing treatment.

3. The apparatus according to claim 1, wherein the reaction region has an inner surface that forms a circular or polygonal frustum, or a concave paraboloid of revolution.

4. The apparatus according to claim 3, wherein a bottom surface area of the reaction region is 1/2 to 1/5 of a top surface area of the reaction region.

5. The apparatus according to claim 4, wherein detection through the nucleic acid amplification reaction is turbidity detection using a LAMP method or a PCR method.

6. A microchip for nucleic acid amplification reaction, comprising:
a reaction region formed as a nucleic acid amplification reaction site, wherein the reaction region is a tapered well of a decreasing cross sectional area along a light axis direction in which a substance that precipitates in the course of a nucleic acid amplification reaction settles.

7. The microchip according to claim 6, wherein the reaction region has a slanted inner surface subjected to a smoothing treatment.

8. A nucleic acid amplification reaction method comprising:
shining light on a reaction region provided as a nucleic acid amplification reaction site, and detecting the quantity of the light scattered by a substance that precipitates in the course of an amplification reaction,
the reaction region being a tapered well of a decreasing cross sectional area along a light axis direction in which the substance that precipitates in the course of the nucleic acid amplification reaction settles.
